(19) United States
(12) Patent Application Publication
(10) Pub. No.: US 2007/0041506 A1
(43) Pub. Date: Feb. 22, 2007

(54) Digital sensor holder

(76) Inventor: Michelle Bottino, Carol Stream, IL (US)

Correspondence Address: Zachary Hamilton, 3340 E. Forestview Trail, Crete, IL, 60417, US

(21) Appl. No.: 11/208,501

(22) Filed: Aug. 22, 2005

(51) Int. Cl. A61B 6/14; G03B 42/02; G03C 5/16

(52) U.S. Cl. 378/170

(57) ABSTRACT

A holder for a dental x-ray image sensor including a handle having a grooved top portion, and a first end, and a L-shaped member having a grooved top portion and a long end. The first end of the handle connects to the long end of the L-shaped member. The grooved top portion of the handle aligns with the grooved top portion of the L-shaped member. Also, there is a bite member, attached adjacent to the long end of the L-shaped member and perpendicular to the long end of the L-shaped member. Wherein, the holder is made of material that glows in the dark.

### RELATED ART

The present invention relates generally to a holder for a dental X-ray image sensor. More specifically, the present invention relates to a device for holding a dental X-ray image detection device for holding Digital Intra Oral Sensors.

### BACKGROUND

Holders for dental X-ray film packets are known in the art. Two examples can be found in U.S. Pat. Nos. 5,256,982 and 4,075,494. The holders disclosed in these patents include a handle to assist the technician in correctly placing the film packets in the patient's mouth. However, for a variety of reasons, such X-ray film holders are cumbersome and uncomfortable for the patient. Bite-wing loops include a bite surface and a rigid paper-like flat loop for accommodating the X-ray packets.

Despite the longstanding popularity of bite-wing loops, there are many disadvantages with their use. For example, the lack of a handle makes them difficult for the technician to place in the mouth correctly. Further, the rigid connection between the X-ray film packet and the loop makes it difficult to adjust the position of the packet depending upon the patient's intraoral anatomy. Specifically, the architecture of patients' upper palate and base of the tongue will vary and the inability of the traditional bite-wing or rigid holding devices to accommodate for the variances between the architectures of individuals' upper palate and tongue area adversely affects the quality of the X-rays.

Further, and more important, the traditional X-ray film packets are being replaced by digital X-ray sensors. Digital X-rays are quickly replacing traditional X-rays in the dental field because less radiation is used and images can be more quickly analyzed and manipulated using a computer.

However, problems have arisen with the use of digital X-ray sensors because they tend to be large and rigid, like traditional X-ray film packets and, an accommodation must be made for the wire lead connected to the sensor. Still further, the inability of traditional bite-wing or traditional holding devices to accommodate for variations in the individual patient's intraoral anatomy is especially problematic with the use of digital X-ray sensors because of their size and rigidity.

Accordingly, there is a need for an improved holding device for digital X-ray sensors that overcomes the deficiencies of the prior art discussed above.

### BRIEF SUMMARY

A dental X-ray image digital sensor holder for holding Digital Intra Oral Sensors aids in the positioning in the mouth of these sensors for X-rays. Furthermore, the digital sensor holder is designed to hold the various sizes and brands of sensors on the market today. The advantage of this new holder of the current holders is that it “glows in the dark”. Placing digital sensors in the oral cavity can be difficult because the mouth is dark.

As stated above, the DSH 100 glows in the dark, which may make placing the digital sensors, aiming and taking the x-ray easier because the target area will be slightly illuminated. Being able to better see the target by using the DSH 100 should eliminate some commons errors produced in normal dental x-rays, such as cone cutting.

In one aspect, the digital sensor holder has a handle having a grooved top portion and is connected to the long side of a L-shaped member that also has a grooved top portion. There is a bite area that is attached adjacent and perpendicular to the long side of the L-shaped member. The handle are attached such that the grooves are aligned. The grooves are used to hold the digital intra oral sensors. Furthermore, the holder glows in the dark realizing the advantages stated above.

In another aspect, the digital sensor holder has a handle having a grooved top portion and is connected to the bottom of a short side of a L-shaped member that also has a grooved top portion. The grooved top portion of the L-shaped member and the grooved top portion of the handle face in the same direction. The grooves are used to hold the digital intra oral sensors. Furthermore, the holder glows in the dark realizing the advantages stated above.

Additional embodiments and advantages of the present invention will become apparent from the following description and the appended claims when considered with the accompanying drawings.

### BRIEF DESCRIPTION OF THE DRAWINGS

FIG. 1 shows a perspective view of an embodiment of a dental X-ray image digital sensor holder, according to the present invention:

FIG. 2 shows a perspective view of an embodiment of a dental X-ray image digital sensor holder, according to the present invention;

FIG. 3 shows a perspective view of an embodiment of a dental X-ray image digital sensor holder, according to the present invention; and

FIG. 4 shows a perspective view of an embodiment of a dental X-ray image digital sensor holder, according to the present invention.

### DETAILED DESCRIPTION OF THE INVENTION

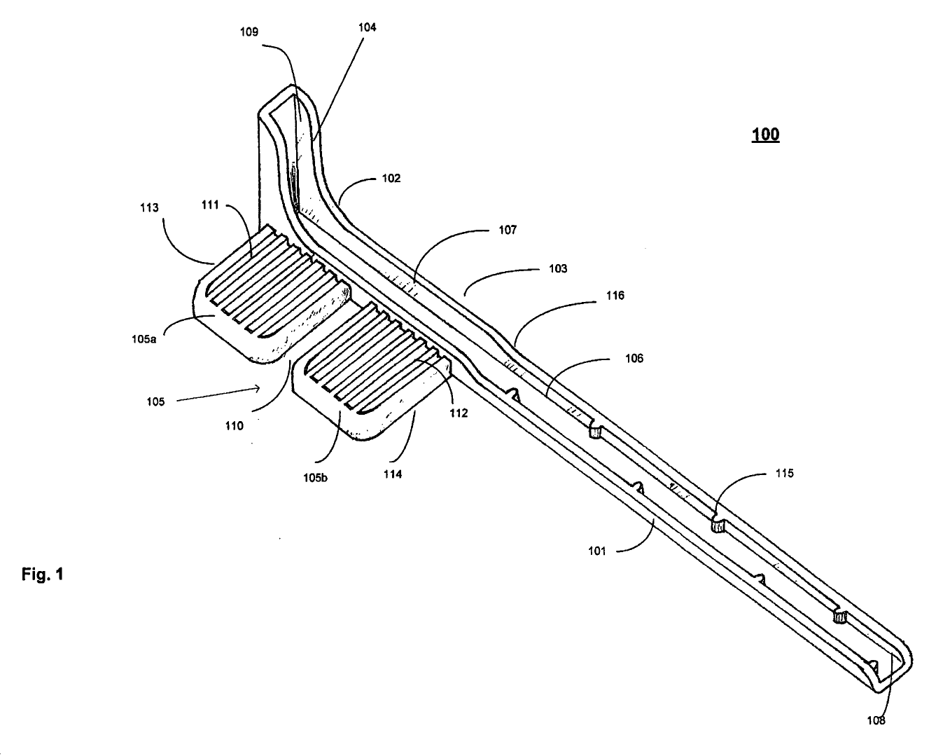

FIG. 1 shows a perspective view of an embodiment of a dental X-ray image digital sensor holder (“DSH”) 100, according to the present invention. The DSH 100 includes a handle 101, a L-shaped member 102 having a long end 103 and a short end 104. The long end 103 of L-shaped member 102 is attached to a end 116 of handle 101, The DSH 100 also includes a bite member 105 attached adjacent to and perpendicular to the long end 103 of L-shaped member 102. The DSH 100 glows in the dark and may reduce the need of dentists, hygienists and other dental staff who may place the DSH 100 in a mouth to do so correctly without turning room lights on and off while x-raying the mouth of a patient.

The handle 101 and the L-shaped member 102 of the DSH 100 include grooves 106 and 107 respectively. The handle 101 and the L-shaped member 102 are attached in such a way that the grooves 106 and 107 align. Handle 101 also has an access end 108 that is open. Likewise, the L-shaped member 102 has an access end 109 that is open.

As stated above, the DSH 100 also includes a bite member 105 attached to and perpendicular to the long end 103 of L-shaped member 103. In the present invention, the bite member 105 includes bite members 105a and 105b, as shown in FIG. 1. Bite members 105a and 105b maybe separated by a space 110. Bite members 105a and 105b may also include ridges 111, 112 on a top side and ridges 113, 114 (not shown) respectively on a bottom side to allow the patient to grip the bite members 105a and 105b better.

Typically, DSH 100 is made of a one piece. In the present invention, the DSH 100 is made of infused glow in the dark autoclavable plastic. A description of the DSH 100 use is described below.

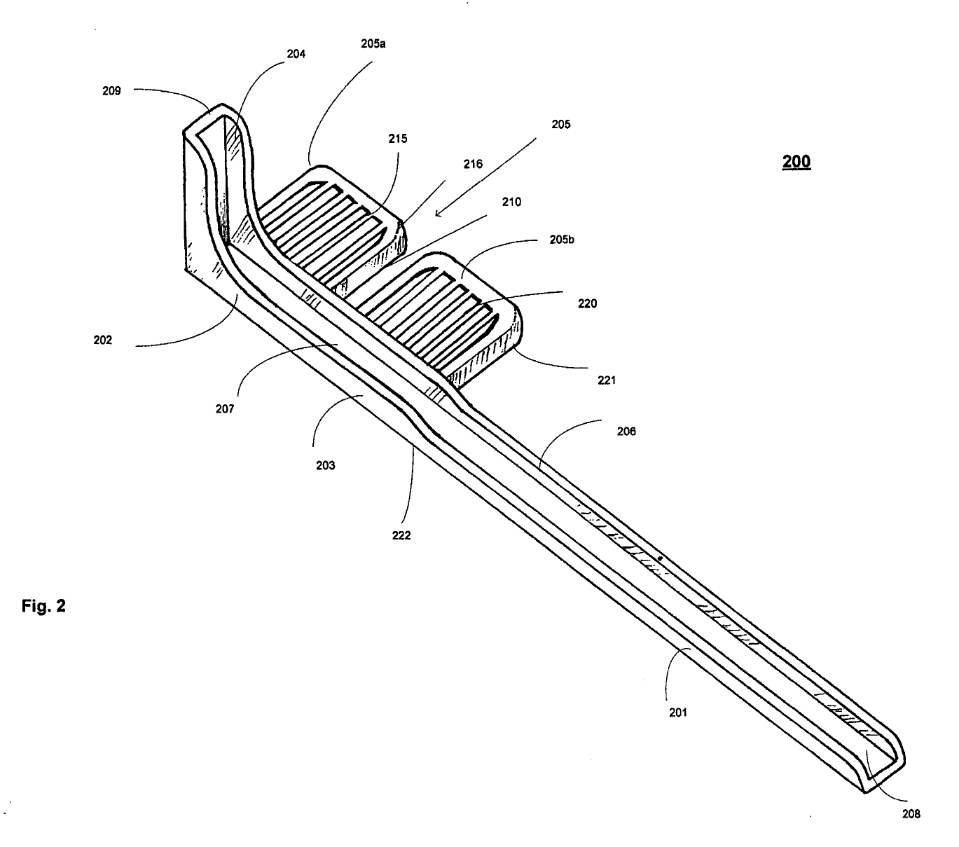

FIG. 1 shows an embodiment of the DSH 100 that is placed on the left side of a face and in the mouth of a patient so that the bite members 105a and 105b may be bitten by the patient's back teeth. The bite members 105a are attached to a left side of the L-shaped member 102 as looking from the handle end 108. A digital intra oral sensor (not shown) is placed within the grooves 106 and 107. The grooves 106 and 107 are used to hold the digital intra oral sensor in place. In the present embodiment, hooks 115 also facilitate holding the digital intra oral sensor in place. However, typically the hooks 115 are not necessary to hold the digital intra oral sensor in place, as shown in FIG. 2.

The digital intra oral sensor can be linked to a computer (not shown).

With the DSH 100 in place within the mouth a digital x-ray can be taken. Typically, x-rays are taken in a darken room. Placing digital sensors in the oral cavity can be difficult because the mouth is dark. As stated above, the DSH 100 glows in the dark, which may make placing the digital senors, aiming and taking x-ray easier because the target area will be slightly illuminated. Being able to better see the target by using the DSH 100 should eliminate some commons errors produced in normal dental x-rays, such as cone cutting.

FIG. 2 shows a perspective view of another embodiment of a dental X-ray image digital sensor holder (“DSH”) 200, according to the present invention. The DSH 200 includes a handle 201, a L-shaped member 202 having a long end 203 and a short end 204. The long end 203 of L-shaped member 202 is attached to a end 222 of handle 201, The DSH 200 also includes a bite member 205 attached adjacent to and perpendicular to the long end 203 of L-shaped member 202. The DSH 200 glows in the dark. Placing digital sensors in the oral cavity can be difficult because the mouth is dark. As stated above, the DSH 200 glows in the dark, which may make placing the digital sensors, aiming and taking the x-ray easier because the target area will be slightly illuminated. Being able to better see the target by using the DSH 200 should eliminate some commons errors produced in normal dental x-rays, such as cone cutting.

The handle 201 and the L-shaped member 202 of the DSH 200 include grooves 206 and 207 respectively. The handle 201 and the L-shaped member 202 are attached in such a way that the grooves 206 and 207 align. Handle 201 also has an access end 208 that is open. Likewise, the L-shaped member 202 has an access end 209 that is open.

As stated above, the DSH 200 also includes a bite member 205 attached to and perpendicular to the long end 203 of L-shaped member 203. In the present invention, the bite member 205 includes bite members 205a and 205b, as shown in FIG. 2. Bite members 205a and 205b are separated by a space 210. Bite members 205a and 205b may also include ridges 220 on a top side and ridges 221 (not shown) on a bottom side to allow the patient to grip the bite members 205a and 205b better.

Typically, DSH 200 is made of a one piece. In the present invention, the DSH 200 is made of infused glow in the dark autoclavable plastic. A description of the DSH 200 use is described below.

FIG. 2 shows an embodiment of the DSH 200 that is placed on the right side of a face and in the mouth of the patient so that the bite members 205a and 205b may be bitten by a patient's back teeth. The bite members 205a are attached to a right side of the L-shaped member 202 as looking from the handle end 208. A digital intra oral sensor (not shown) is placed within the grooves 206 and 207. The grooves 206 and 207 are used to hold the digital intra oral sensor in place.

The digital intra oral sensor can be linked to a computer (not shown).

With the DSH 200 in place within the mouth a digital x-ray can be taken. Typically, x-rays are taken in a darken room. Placing digital sensors in the oral cavity can be difficult because the mouth is dark. As stated above, the DSH 200 glows in the dark, which may make placing the digital senors, aiming and taking the x-ray easier because the target area will be slightly illuminated. Being able to better see the target by using the DSH 200 should eliminate some commons errors produced in normal dental x-rays, such as cone cutting.

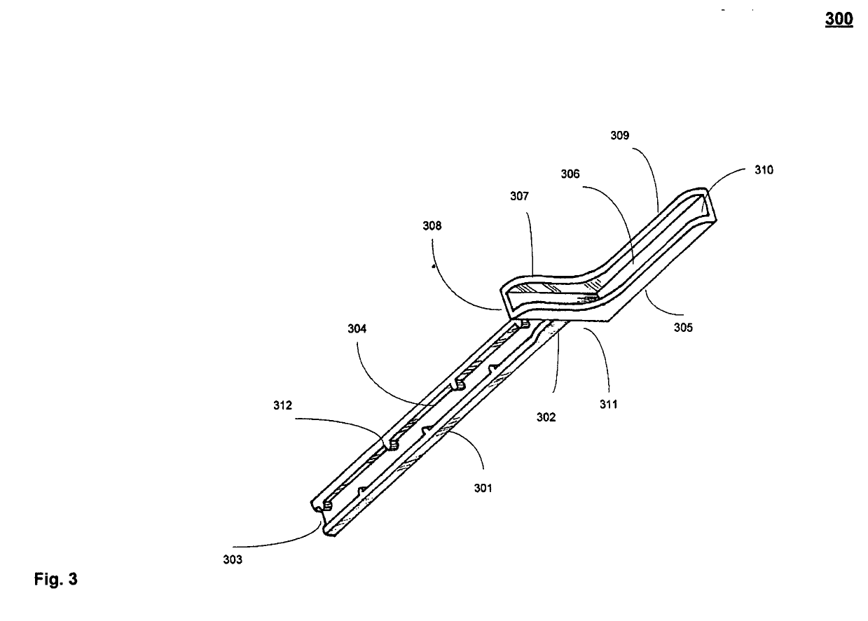

FIG. 3 shows a perspective view of another embodiment of a dental x-ray image dental sensor holder (“DSH”) 300 includes a handle 301 having a end 302 an access end 303, and a groove 304, a L-shaped member 305 having a groove 306 on an interior of the L-shaped member 305, a short portion 307 having end 308, and a long portion 309 having and end 310. The end 302 of the handle 301 is connected to an underside 311 of the short portion 307 of the L-shaped member 305, typically, near the center of the short portion 307.

In the present invention, the DSH 300 is made of infused glow in the dark autoclavable plastic, and is typically cast as on piece. A description of the DSH 100 use is described below.

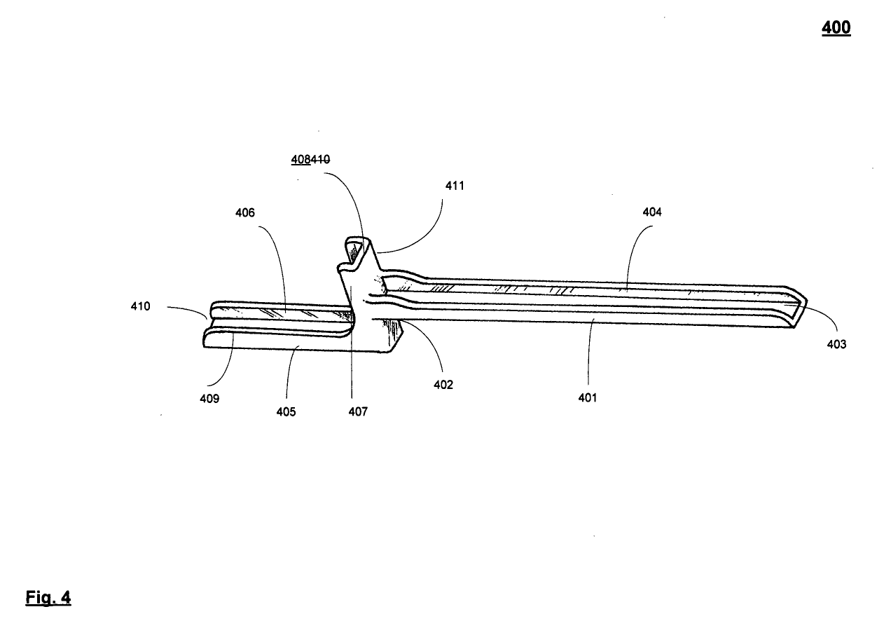

A digital intra oral sensor (not shown) is placed within groove 306 and bent over 308 and into groove 304 and out of access end 303. The digital intra oral sensor can be linked to a computer (not shown). Grooves 308 and 304 are designed to accommodate a variety of digital intra oral sensors. However, typical digital intra oral sensors may not require hooks 312, to hold them within the groove 304 of the handle 301, as shown in FIG. 4.

The DSH 300 is place in the front of a mouth and is held secure by having a patient bite down on the short portion 307. With the DSH 300 in place within the mouth a digital x-ray can be taken. Typically, x-rays are taken in a darken room. Placing digital sensors in the oral cavity can be difficult because the mouth is dark. As stated above, the DSH 300 glows in the dark, which may make placing the digital senors, aiming and taking the x-ray easier because the target area will be slightly illuminated. Being able to better see the target by using the DSH 300 should eliminate some commons errors produced in normal dental x-rays, such as cone cutting.

FIG. 4 shows a perspective view of another embodiment of a dental x-ray image dental sensor holder (“DSH”) 400 includes a handle 401 having a end 402 an access end 403, and a groove 404, a L-shaped member 405 having a groove 406 on an interior of the L-shaped member 405, a short portion 407 having end 408, and a long portion 409 having and end 410. The end 402 of the handle 401 is connected to an underside 411 of the short portion 407 of the L-shaped member 405, typically, near the center of the short portion 407.

In the present invention, the DSH 400 is made of infused glow in the dark autoclavable plastic, and is typically cast as on piece. A description of the DSH 400 use is described below.

A digital intra oral sensor (not shown) is placed within groove 406 and bent over 408 and into groove 404 and out of access end 403. The digital intra oral sensor can be linked to a computer (not shown). Grooves 408 and 404 are designed to accommodate a variety of digital intra oral sensors.

The DSH 400 is place in the front of a mouth and is held secure by having a patient bite down on the short portion 407. With the DSH 400 in place within the mouth a digital x-ray can be taken. Typically, x-rays are taken in a darken room. Placing digital sensors in the oral cavity can be difficult because the mouth is dark. As stated above, the DSH 400 glows in the dark, which may make placing the digital sensors, aiming and taking the x-ray easier because the target area will be slightly illuminated. Being able to better see the target by using the DSH 400 should eliminate some commons errors produced in normal dental x-rays, such as cone cutting.

The foregoing detailed description is merely illustrative of several physical embodiments of the invention. Physical variations of the invention, not fully described in the specification, may be encompassed within the purview of the claims. Accordingly, any narrower description of the elements in the specification should be used for general guidance, rather than to unduly restrict any broader descriptions of the elements in the following claims.

What is claimed is:

1. A holder for a dental X-ray image sensor comprising:
a handle comprising a grooved top portion, and a first end;
a L-shaped member comprising a grooved top portion, and a long end;
said first end of handle connected to said long end of L-shaped member,
wherein said grooved top portion of said handle aligns with said grooved top portion of said L-shaped member; and
a bite member, attached adjacent to said long end of said L-shaped member and perpendicular to said long end of said L-shaped member,
wherein said holder glows in the dark.

2. The holder of claim 1 wherein said bite member comprising a first bite member and a second bite member.

3. The holder of claim 1 wherein said bite member has ridges on a top side.

4. The holder of claim 1 wherein said bite member has ridges on a bottom side.

5. The holder of claim 2 wherein said first bite member and said second bite member are separated by a space.

6. The holder of claim 3 wherein said first bite member and said second bite member are attached to a left side of said long end of said L-shaped member.

7. The holder of claim 3 wherein said first bite member and said second bite member are attached to a right side of said long end of said L-shaped member.

8. The holder of claim 1 wherein said holder is made of infused glow in the dark autoclavable plastic.

9. The holder of claim 1 further comprising hooks disposed within said grooved portion of said handle.

10. A holder for a dental X-ray image sensor comprising:
a handle comprising a grooved top portion, a first end and a second end;
a L-shaped member comprising a grooved interior, and a short end;
said first end of handle connected to a bottom of said short end of L-shaped member; and
wherein said grooved top portion of said handle and said grooved top portion of said L-shaped member face in the same direction, and
wherein said holder glows in the dark.

11. The holder of claim 10 wherein said holder is comprised of infused glow in the dark autoclavable plastic.

12. The holder of claim 10 further comprising hooks disposed within said grooved portion of said handle.
